(19) United States
(12) Patent Application Publication
(10) Pub. No.: US 2015/0080738 A1
(43) Pub. Date: Mar. 19, 2015

(54) APPARATUS, METHOD, AND COMPUTER-ACCESSIBLE MEDIUM FOR DETERMINING ANTIGEN IMMUNOREACTIVITY IN TISSUE

(71) Applicants: GUillermo J. Tearney, Cambridge, MA (US); Brett Eugene Bouma, Quincy, MA (US); Aubrey Katz, Newton, MA (US); Wayne Shreffler, Beverly, MA (US); Qian Yuan, Sharon, MA (US)

(72) Inventors: GUillermo J. Tearney, Cambridge, MA (US); Brett Eugene Bouma, Quincy, MA (US); Aubrey Katz, Newton, MA (US); Wayne Shreffler, Beverly, MA (US); Qian Yuan, Sharon, MA (US)

(73) Assignee: The General Hospital Corporation, Boston, MA (US)

(21) Appl. No.: 14/239,410

(22) PCT Filed: Aug. 16, 2012
(86) PCT No.: PCT/US2012/051132

§ 371 Date: Nov. 12, 2014

Related U.S. Application Data
(60) Provisional application No. 61/524,207, filed on Aug. 16, 2011.

(51) Int. Cl. A61B 5/00; A61B 1/307; A61B 1/313

(52) U.S. Cl. CPC A61B 5/411; A61B 1/3132; A61B 1/307; A61B 5/0066; A61B 5/0068
USPC 600/476; 600/300; 600/109

(57) ABSTRACT

Exemplary configurations can be provided which can be structured to be included and/or inserted in an internal anatomical structure. For example, the configuration(s) can have at least one first arrangement which can include at least one substance that, when applied to at least one section of the internal anatomical structure, has a potential to induce an inflammatory response in the section(s). The configuration(s) can also have at least one second (e.g., microscopy) arrangement which can be configured to receive information from the section(s), and determine whether the inflammatory response has occurred therein.

### CROSS-REFERENCE TO RELATED APPLICATION(S)

This application is based upon and claims the benefit of priority from U.S. Patent Application Ser. No. 61/524,207 filed Aug. 16, 2011, the entire disclosure of which is incorporated herein by reference.

### FIELD OF THE DISCLOSURE

Exemplary embodiments of the present disclosure relate to apparatus, method and computer-accessible medium for determining antigen immunoreactivity in tissue

### BACKGROUND INFORMATION

A variety of diseases, such as eczema, asthma, eosinophilic esophagitis, and inflammatory bowel disease, are known or thought to result from an immune reaction to antigens that come in contact with or are present within tissue. In the skin, for example, evidence of the offending antigen can be gained by topical application, epicutaneous injection (prick skin test “PST”) or subcutaneous injection of the antigen and visualizing the reaction that occurs around the deposited antigen. One such test that is commonly used is the so-called PATCH test, in which antigens are applied topically, in an array, where distinct elements of the array contain a different antigen. The patch is typically left on for 48 hours. The patch can then be removed, and a cell-mediated hypersensitivity to the offending antigen creates a local inflammatory response over the affected region where the antigen was deposited. Another common antigen test is a TB or PPD test, which is used to determine if a patient has been exposed to tubercle bacillus. With PPD testing, purified proteins from the bacteria are deposited intradermally, and followed over a period of about 24-72 hours to observe whether or not there is a cell-mediated immune response, exhibited by skin induration and redness at the injection site. In prick testing, e.g., the antigen can be introduced to the epicutaneous layer of the skin, and evidence of an IgE-mediated response can be measured as a wheal (angioedema) and flare (erythema) response with minutes of its application.

While these tests have been useful for certain conditions, various atopic diseases that manifest in internal organs may not be accurately characterized by skin testing. For example, with respect to eosinophilic esophagitis, an allergic disorder of the esophagus, PATCH and PST testing has had limited success in determining the underlying antigen that precipitates the disease. Another possible problem with PATCH testing can be that it takes a long time (e.g., 72 hours) to obtain a reaction that can be definitively determined to be positive. In addition, frequently, the reaction is ambiguous and unclear, likely making it difficult to confirm or deny positivity.

Given these limitations of conventional antigen testing of gastrointestinal forms of allergy such as EoE, it may be desirable to have a more accurate antigen test that could be evaluated at a much earlier time than that required to develop a visible lesion on the skin. It can also be beneficial to provide organ specific antigen testing that can more accurately characterize the offending antigen that affects the particular organ, such as the esophagus, colon, rectum, bladder, pulmonary airways, or the like.

Early stages of cell-mediated or humoral or hypersensitivity reactions can be characterized by cellular changes in tissue, including antigen presenting cell (APC) movement and proliferation, infiltration of leukocytes, macrophages, basophils, lymphocytes, and eosinophils, into the tissue in response to activated APCs. For instance eosinophils are common cellular infiltrates that are elevated during IgE-mediated reactions for many allergic/atopic disease such as EE and asthma. In vivo microscopy is capable of identifying these cells in tissue and within any organ in the body (as shown in FIGS. 1(A)-1(D)). These cells can also be specifically elevated in organs where the disease manifests, such as the esophagus, colon, and pulmonary airways or lung. Furthermore, local immune responses, including the production of IgE, can occur at sites of allergic inflammation regardless of whether those individuals have evidence of allergic responses on skin testing. Thus, visualization and quantification of cellular inflammation using in vivo microscopy imaging devices can provide a far more sensitive way for determining antigen responsiveness and for identifying the offending antigens driving organ-specific atopic disorders.

Accordingly, it may be beneficial to address at least some of the above-described deficiencies by providing sub-diffraction limited imaging of intact or living tissues.

### OBJECTS AND SUMMARY OF EXEMPLARY EMBODIMENTS

Thus, at least some of the above-described issues and/or deficiencies can be addressed with the exemplary embodiments of the systems, methods and computer-accessible medium according to the present disclosure which provide sub-diffraction limited imaging of intact or living tissues.

According to an exemplary embodiment of the present disclosure, systems, methods and computer-accessible medium can be provided for antigen testing that can improve sensitivity of antigen exposure testing and determination of the offending antigen in allergic inflammatory disease by providing a way to facilitate direct antigen testing in the affected internal organs that are the sites of inflammation. For example, it is possible to utilize a topical or intramucosal application of one or a series of different antigens to an external or internal organ. In one embodiment, the antigen can be applied to the tissue. In yet another exemplary embodiment of the present disclosure, a control can be applied. In another exemplary embodiment of the present disclosure, the antigen can be applied topically using an exemplary topical application arrangement, such as a patch. Further or in addition, the antigen can be applied by injecting the antigen into the epithelium, lamina propria, submucosa, muscle, subcutaneous tissue, organ or the like. In still another exemplary embodiment of the present disclosure, an antigen can be topically applied or injected in multiple locations on or within the tissue. In a further exemplary embodiment of the present disclosure, an array of antigens can be applied, each at least one antigen is configured to be deposited at a different spatial location in the tissue. The antigens can be applied topically using an arrayed topical application arrangement, such as a patch, or can be injected using an exemplary array injection arrangement.

For example, the exemplary array injection arrangement according to still another exemplary embodiment of the present disclosure can include an array of needles, where at least one of the needles can contain a different antigen. In one exemplary embodiment of the present disclosure, at least one antigen can be topically applied or injected in skin, other external mucosa and/or an internal organ. In another embodiment, the internal organ is a luminal organ (which can contain an epithelium) and/or an internal organ where the disease symptoms manifest. A tumor substance, protein, cell, or molecule associated with a tumor, which (e.g., a cancerous tumor) can be injected into the subject and monitored via in vivo microscopy to determine if a cell mediated, humoral, or hypersensitivity response occurs. This exemplary response can be indicative of the early stage of spread of this disease or may be used as an exemplary screening device to determine whether or not the subject has cancer.

Instead of visually observing the macroscopic wheal and/or flare immune response to the at least one antigen, according to another exemplary embodiment of the present disclosure, it is possible to use on vivo microscopic imaging to observe the cellular infiltrates that occur in response to the offending antigen. In one further exemplary embodiment, the imaging results can be obtained by a reflectance or fluorescence imaging system, and/or can be conducted following labeling of the specimen with a compound designed to mark a particular molecule on at least one cell. An exemplary imaging technique can include, but not limited to, spectrally-encoded microscopy (SECM), reflectance confocal microscopy, optical coherence tomography, spectral-domain optical coherence tomography, optical frequency domain imaging, fluorescence confocal microscopy, auto fluorescence imaging, auto fluorescence confocal microscopy, two- or three-photon microscopy, and/or second or third harmonic microscopy. In a further exemplary embodiment, the imaging can be conducted (i) in the skin, (ii) in a luminal organ inside the body, including the esophagus, stomach, small intestine, large intestine, trachea, pulmonary airway, urethra, bladder, ureter, sinuses or the like, and/or (iii) using a catheter, probe, laparoscope or endoscope.

In yet another exemplary embodiment of the present disclosure, the imaging system and/or probe can be configured to provide substantially transverse, cross-sectional, and/or three-dimensional image(s). For example, the probe can be a transnasal, transoral, pill and/or tethered pill device, and/or can contains a centering device. The exemplary probe can contain an apparatus that can positions the imaging arrangement at a predetermined distance from the luminal surface. According to a further exemplary embodiment, the exemplary probe can contain a positioning device (e.g., a wire basket or balloon), a pH, motion, and/or pressure sensor. Such exemplary probe can be configured to provide information required to place the antigen application apparatus or imaging arrangement at a predetermined location within the internal organ. In yet another exemplary embodiment the exemplary probe can be in the shape of a swallowable configuration such as a capsule or pill that can be swallowed. In yet a further embodiment, the capsule can be tethered so that the operator can control the position of the capsule over the area where the antigens and or controls have been deposited or applied. In a further embodiment this tether can contain at least one of an optical fiber to transmit light or an electrical connection to transmit an electrical signal, In yet another embodiment the capsule is wireless and transmits information regarding the deposited antigen via a wireless transmission means.

According to another exemplary embodiment of the present disclosure, the exemplary in-vivo microscopy imaging technique/procedure can be used (e.g., with an exemplary computer processing arrangement) to obtain images of cellular infiltrates that can be an eosinophil, a lymphocyte, a plasma cell, a basophil, and/or a mast cell. The imaged cells can include APC's including monocyte, macrophage, Langerhans's cell and/or dendritic cell. For example, these cells can be counted and a threshold can be used to determine antigen responsivity. The cell count can be normalized to an area or volume that is located in proximity to the antigen application site. Further, according to still another exemplary embodiment of the present disclosure, the cells can be counted using a processing arrangement in a substantially automated way. The activity of the cells (i.e. eosinophil degranulation) can be qualitatively evaluated or quantified using the exemplary imaging arrangement.

One of the exemplary advantages of the exemplary embodiments of the present disclosure can be that the observation period for determining responsivity to the antigen can be more rapid than PATCH testing. In certain exemplary embodiments of the present disclosure, the exemplary in vivo microscopy observation of the cellular infiltrates can occur within less than at least about 15 minutes, 30 minutes, 1 hr, 2 hrs, 3 hrs, etc. following the application of the antigen(s). For example, in another exemplary embodiment, the observation period can be at least approximately less than about 6, 12, or 24 hours.

Other diseases, such as cancer and autoimmune disease, can also be typified by a host response to cancer antigens via cell mediated or humoral immunity. These diseases can be detected at an early stage by injection of autoantigens or tumor antigens and observation by in vivo microscopy. Cancerous conditions can also be staged to determine whether or not they are in situ or invasive based on their ability to have elicited an immune response at a remote site, observed by in vivo microscopy, following the injection or patch application of the autoantigen or tumor antigen substance.

According to certain exemplary embodiments of the present disclosure, configurations can be provided which can be structured to be included or inserted in an internal anatomical structure. For example, the configuration(s) can have at least one first arrangement which can include at least one substance that, when applied to at least one section of the internal anatomical structure, has a potential to induce an inflammatory response in the section(s). The configuration(s) can also have at least one second (e.g., microscopy) arrangement which can be configured to receive information from the section(s), and determine whether the inflammatory response has occurred therein.

According to one exemplary embodiment, the first arrangement(s) can have an array of portions into which a plurality of the substances are provided. The first arrangement(s) can be structured to be placed on the surface of the section (or within the section(s), and can be configured to be injected into the section(s). The second arrangement(s) can include a video-imaging probe, and can be an in vivo arrangement. The vivo-microscopy arrangement can be a spectrally-encoded microscopy (SECM) arrangement, a reflectance confocal microscopy arrangement, an optical coherence tomography arrangement, a spectral-domain optical coherence tomography arrangement, an optical frequency domain imaging arrangement, a fluorescence confocal microscopy arrangement, an auto fluorescence imaging arrangement, an auto fluorescence confocal microscopy arrangement, a two-photon microscopy arrangement, a three-photon microscopy arrangement, a second harmonic microscopy arrangement, and/or a third harmonic microscopy arrangement.

In another exemplary embodiment, the first arrangement(s) or the second arrangement(s) can include a balloon, a wire basket and/or a capsule having a shape that is configured to be swallowed. The substance(s) can include a tumor substance, protein, cell, and/or molecule associated with a tumor. The second arrangement(s) can be configured to determine if a cell mediated response, a humoral response, and/or a hypersensitivity response which occurs based on the inflammatory response. The second arrangement(s) can further be configured to generate at least one image of the section(s) using the information. The image can include a two-dimensional image and/or a three-dimensional image.

According to still another exemplary embodiment of the present disclosure, the second arrangement(s) can also be configured to determine whether the information is regarding at least one particular cell. The particular cell(s) can include an eosinophil, a lymphocyte, a plasma cell, a basophil, a mast cell, a monocyte, a macrophage, a Langerhans's cell, and/or a dendritic cell, and the second arrangement can be further configured to quantify a number of the cell(s). In addition, the second arrangement can be further configured to utilize a threshold of the number to determine antigen responsiveness of the section(s) Further, the inflammatory response can be a hypersensitivity, a cell mediated immunity and/or a humoral immunity.

These and other objects, features and advantages of the present invention will become apparent upon reading the following detailed description of embodiments of the disclosure, when taken in conjunction with the appended claims.

### BRIEF DESCRIPTION OF THE DRAWINGS

Further objects, features and advantages of the present disclosure will become apparent from the following detailed description taken in conjunction with the accompanying drawings showing illustrative embodiments of the present disclosure, in which:

FIG. 1(A) is an exemplary SECM image of an esophageal biopsy specimen, obtained at 50 μm from the surface, demonstrating a diffuse infiltration of highly scattering cells at low magnification;

FIG. 1(B) is an exemplary illustration of higher magnification view of a light dotted region illustrated in FIG. 1(A) shows that these cells are eosinophils with bi-lobed nuclei (inset);

FIG. 1(C) is an exemplary Histology of eosinophil degranulation that is indicative of active eosinophilic inflammation (scale bars being 100 μm);

FIG. 1(D) is an exemplary SECM image associated with that of FIG. 1(A);

FIG. 2 is a diagram of an apparatus configured to inject at least one different antigen or antigen concentration at a distinct location in the specimen according to an exemplary embodiment of the present disclosure;

FIG. 3 is a diagram of an array of antigens applied to the specimen using at least one of a patch apparatus or a needle apparatus according to an exemplary embodiment of the present disclosure;

FIG. 4 is a diagram of an endoscope apparatus configured to apply a patch apparatus containing at least one antigen or a plurality of different antigens on an internal anatomical structure according to an exemplary embodiment of the present disclosure;

FIG. 5 is a diagram of the endoscope apparatus configured to inject a plurality of different antigens on an internal anatomical structure according to another exemplary embodiment of the present disclosure;

FIG. 6 is a diagram of the endoscope apparatus configured to image an array of applied antigens within an internal anatomical structure according to still another exemplary embodiment of the present disclosure; and

FIG. 7 is a diagram of a swallowable capsule apparatus configured to image an array of applied antigens within an internal anatomical structure according to yet another exemplary embodiment of the present disclosure.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject disclosure as defined by the appended claims.

### DETAILED DESCRIPTION OF EXEMPLARY EMBODIMENTS

According to an exemplary embodiment of the present disclosure, at least one antigen or a plurality of antigens, including molecules, proteins, bacteria, viruses, or the like can be applied to an anatomical structure. Following the exemplary application of antigens, an exemplary imaging procedure can be conducted to observe the microstructural changes in the anatomical structure that are associated with the deposition of the antigens. In an exemplary embodiment of the present disclosure, the exemplary imaging procedure can include a microstructural and/or molecular imaging, including RCM, SECM, OCT, SD-OCT, μOCT, OFDI, fluorescence confocal, confocal laser endomicroscopy, multiphoton, second or third harmonic, auto fluorescence, and/or the like. These exemplary in vivo microscopy imaging technologies can be implemented via an endoscopic catheter, probe, or miniaturized imaging system that can be inserted into an organ of interest.

In yet another exemplary embodiment of the present disclosure, the imaging of the antigen can be accomplished using an endoscopy or endoscopic microscopy configuration/apparatus that can be present within a capsule or pill that may be swallowed. The pill may contain a tether to control its position in the organ or it may be wireless and transmit image information outside the body using a wireless transmission configuration that can emit, for example, RF electromagnetic radiation. In yet a further exemplary embodiment of the present disclosure, the wireless capsule may be powered by a battery.

For those antigens that cause a cellular-immunity or hypersensitivity reaction, the imaging modality shows images that correspond to an influx of inflammatory cells, including at least one of a lymphocyte, T-cell, B-cell, plasma cell, eosinophil, basophil, macrophage, monocyte, or the like. In a further exemplary embodiment, the imaging modality can display information of inflammatory cell activity, which may be direct, including at least one of a degranulation, antigen presentation, cytokine expression, chemokine expression, RNA transcription, or the like. In yet another exemplary embodiment, the responses to the deposited substances can be observed by macroscopic imaging technology such as white-light endoscopy, narrow band imaging, chromoendoscopy, or magnification endoscopy or the like.

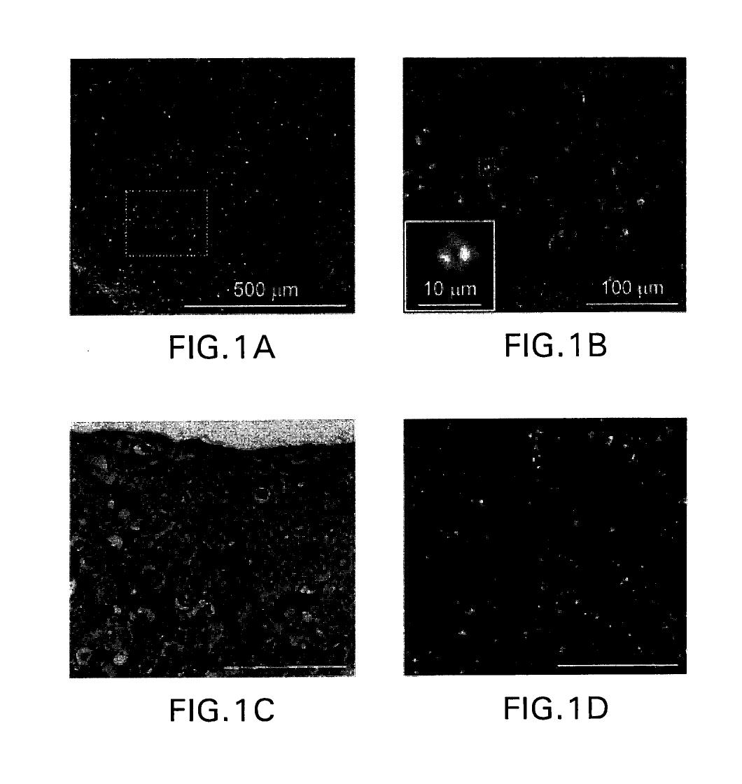

One example of such imaging results is shown in FIGS. 1(A)-1(C), which depict eosinophils in esophageal tissue specimen. These exemplary images were acquired with SECM technique(s), which can be a reflectance microscopy technique that can be configured to obtain images within luminal organs of the body such as the esophagus. Eosinophils by exemplary SECM appear as bright cells, e.g., sometimes with bi-lobed nuclei. Degranulation of eosinophils can also be observed (as shown in FIG. 1(D)). According to an exemplary embodiment of the present disclosure, exemplary SECM technique(s) can be used to identify IgE mediated reactions around the deposited antigen that is responsible for hypersensitivity.

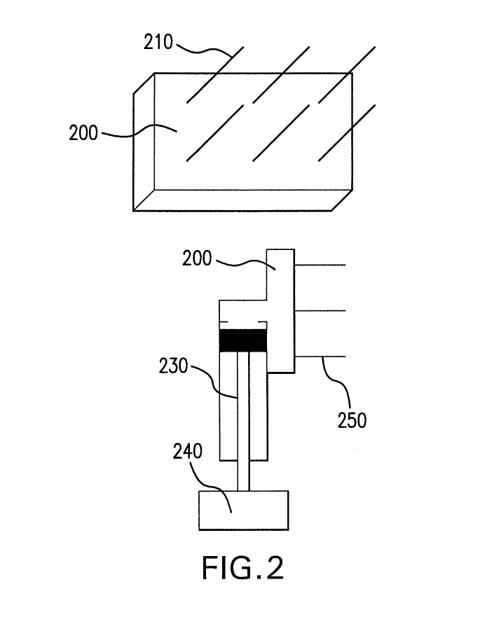

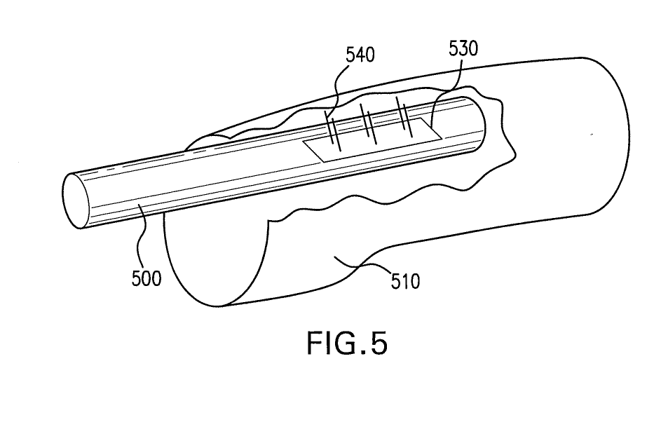

In an exemplary embodiment for injection of at least one antigen or a plurality of antigens, a needle device can be used. The exemplary needle device (e.g., a needle) can be used to inject, e.g., single antigens in the anatomical structure. In another exemplary embodiment, as shown in FIG. 2, a device 200 comprising an array of needles 210, 530 can be used. The exemplary device 200 can be preloaded with one or more than one antigens using, e.g., a syringe device 230. A plunger can be used to withdraw antigens from a well. The exemplary device 200 can be positioned so that needles 250, 540 (as shown in FIGS. 2 and 5, respectively) can penetrate the anatomical structure, and the syringe plunger 240 can be pushed to inject at least one antigen within the tissue of the anatomical structure. The location of tips of the needles 240, 540 can be within the epithelium, lamina propria, submucosa, or any other tissue below the luminal or other surface of the anatomical structure. The exemplary device 200 can be implemented using an endoscopic needle based apparatus, which can be inserted into a natural orifice using and endoscopic device 500 (shown in FIG. 5) or used externally to apply at least one antigen. The anatomical structure can be a luminal tissue such as an esophagus, colon, small bowel, rectum, airway or the like. The anatomical structure can also be the skin.

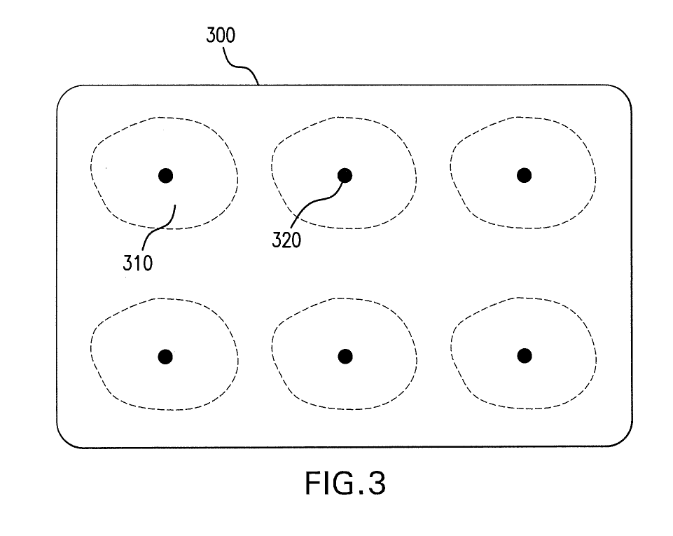

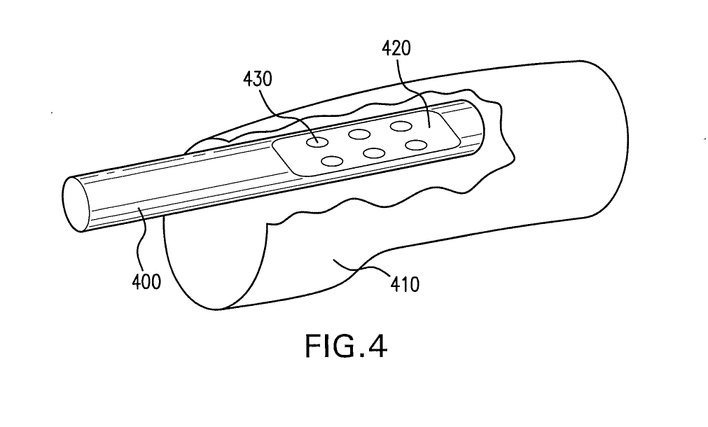

In another exemplary embodiment of the present disclosure, at least one antigen can be applied in an array on the anatomical structure using a patch 300,420 that contains the antigens 310, 320, 430 (as shown in FIGS. 3 and 4, respectively), where each antigen can be provided at a different spatial location. An exemplary endoscopic device 400 (shown in FIG. 4) can be inserted into a luminal organ to apply the patch. The patch 300, 420 can configured to be applied to the anatomical structure in such a manner that the antigens 310, 320, 430 come off the patch 300, 420, and become deposited on the anatomical structure. The antigens 310, 320, 430 can be combined with another substance, such as DMSO, to enhance permeability in the epithelial tissue. In still another exemplary embodiment of the present disclosure, the antigens 310, 320, 430 can also be applied within the tissue of the anatomical structure using an exemplary needle apparatus, such as a single needle or the needle array apparatus of device 200 depicted in FIG. 2.

Following an exemplary application of at least one antigen 310, 320, 430, a period of time can elapse, such as, e.g., about 15, 30, 45 or 60 minutes. Alternatively or in addition, at least less than about 1, 2, 4 hours can elapse or no longer than about 12 or 24 hours. An exemplary in vivo microscopy apparatus configured to image the tissues at a molecular or cellular scale can then be applied to visualize the region on the anatomical structure wherein the at least one antigen was applied. Qualitative or quantitative image analysis techniques and/or procedures, such as blob counting, intensity metrics, or the like can be applied (e.g., by a configured computer arrangement) to the images to determine whether or not a reaction has occurred at one or more of the sites where at least one antigen was deposited. An exemplary scoring procedure can be applied (e.g., by a configured computer arrangement) to the images to determine positivity. A positive reaction can denote at least one antigen or more than one antigens that the patient has been previously exposed to or that causes the patient's hypersensitivity inflammatory reaction.

In yet another exemplary embodiment of the present disclosure, the exemplary device can be used to detect an early cancer, in one embodiment via humoral autoimmunity to tumor antigens, and in still another exemplary embodiment, via cell mediated immune response to tumor antigens. In such exemplary embodiment(s), cells, tissue, mortarized tissue, proteins, DNA, or other molecules or the like associated with a tumor cell can be injected into the subject, in the skin, epidermis, dermis, subcutaneous tissue, or into another organ of the body. The subject can undergo an immune response to the injected material, which can be monitored via in vivo microscopy in internal or external organ systems as described above. The immune response, be it humoral, cell mediated, or hypersensitivity, can be qualitatively analyzed or quantified as described herein by using, e.g., the exemplary in vivo microscopy to visualize the cellular response to the injected substance (e.g., in one exemplary embodiment as a function of time) to determine if the subject has a cancer, and/or if the cancer has begun to spread to the extent that its antigens have caused or accelerated or otherwise facilitated a certain immune response in proximity to the site of injection of the tumor associated substance.

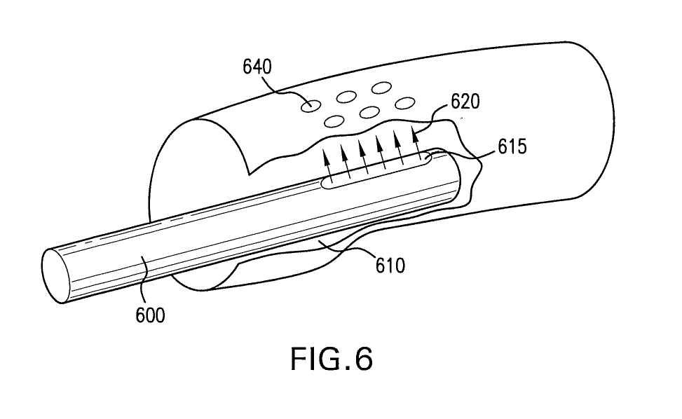

According to a further exemplary embodiment of the present disclosure, the readout of the antigen reaction can be accomplished via endoscopic device which can be used internally or externally to apply at least one antigen and thereafter, electro-magnetic radiation. FIG. 6 depicts a schematic diagram of such exemplary device. For example, as shown in FIG. 6, the antigens 615 introduced into the organ of interest 610. The electro-magnetic radiation (e.g., light) 620 from optics within the capsule 760 can be directed toward the deposited or injected antigen(s) 640, and scanned across the region of antigen deposition. The exemplary device can be wireless or tethered, connected to the outside via a string or a sheath comprised of fiber optics, electrical wires or the like via the endoscopic device 600. Either or both the antigen depositing device or the information gathering detection device can have a position tracking configuration associated with it so as to know where the antigen deposition took place within the organ or to know where to image the response to the antigen within the internal organ.

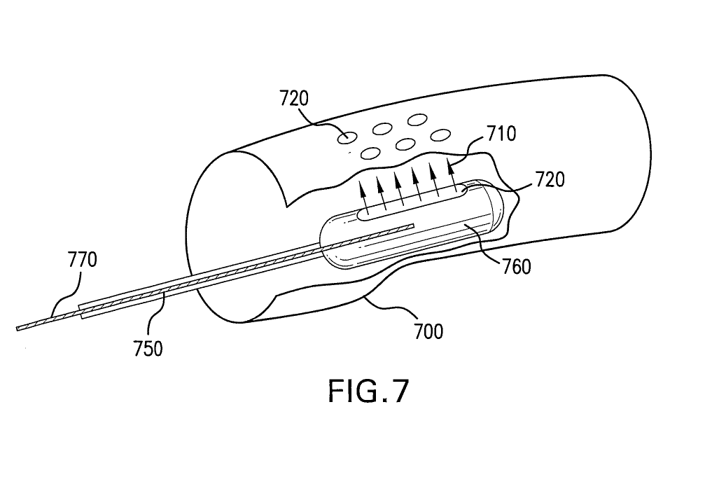

In a further exemplary embodiment of the present disclosure, the readout of the antigen reaction can be accomplished via a capsule device that can be configured, structured and/or sized to be swallowed. FIG. 7 depicts a schematic diagram of such exemplary device. For example, as shown in FIG. 7, following an application of the antigen, a capsule or “pill” 760 that can be configured to readout the reaction to the deposited antigens 720 can be swallowed, inserted, and/or otherwise introduced into the organ of interest 700. Electro-magnetic radiation (e.g., light) 710 from optics within the capsule 760 can be directed toward the deposited or injected antigen(s) 720, and scanned across the region of antigen deposition. The exemplary device can be wireless or tethered, connected to the outside via a string or a sheath comprised of fiber optics, electrical wires or the like 750. The sheath can also contain a driveshaft 770 that can, according to one exemplary embodiment, enclose the fiber or electrical components and that can facilitate internal components to translate, move or otherwise rotate, which can rotate and/or move components within the capsule. In yet another exemplary embodiment, the applicator that applies the antigen or array of antigens can be configured to be in the shape of a pill so that it may be swallowed. Either or both the antigen depositing device or the information gathering detection device may have a position tracking configuration associated with it so as to know where the antigen deposition took place within the organ or to know where to image the response to the antigen within the internal organ.

It is possible that there may be a situation where the antigen does not penetrate the mucosal barrier. If this occurs, the antigen can be mixed with a tissue penetrating agent such as DMSO or the like to enhance penetration of the antigen into the tissue. It also is possible that when the antigen becomes in contact with the tissue, it may disperse spatially making localization by in vivo microscopy challenging. In order to minimize the antigen spatial dispersion in the tissue, it can be mixed with a substance that serves to at least partially maintain the antigen(s) localized spatially on or within the tissue, such as viscous substances of sucrose, dextran, or the like.

In another exemplary embodiment of the present disclosure, exemplary systems, methods and computer-accessible medium can be provided to localize the regions associated with the application of the offending antigen and/or controls. In one exemplary embodiment, an ink can be applied on or near the region of interest, demarcating the locations of the applied antigens. In a further exemplary embodiment, the ink can be applied in the mucosa to tattoo the tissue so as to facilitate a follow up of the sites over a duration of time. In still another embodiment, a substance such as indocyanine green, methlyne blue, trypan blue, Lugol's or the like can be applied to the tissue. This exemplary marker can also be visualized by the exemplary in vivo microscopy technique/procedure to facilitate a registration of the exemplary in vivo microscopy images and the sites in which the antigens were applied.

The foregoing merely illustrates the principles of the present disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, U.S. Patent Publication No. 2002/0122246, published on May 9, 2002, U.S. Patent Application No. 61/649,546, U.S. patent application Ser. No. 11/625,135, and U.S. Patent Application No. 61/589,083, the disclosures of which are incorporated by reference herein in their entireties. It will thus be appreciated that those skilled in the art will be able to devise numerous systems, arrangements and methods which, although not explicitly shown or described herein, embody the principles of the present disclosure and are thus within the spirit and scope of the present disclosure. Further, various exemplary embodiments described herein can be interchangeably used with all other exemplary described embodiments, as should be understood by those having ordinary skill in the art. In addition, to the extent that the prior art knowledge has not been explicitly incorporated by reference herein above, it is explicitly being incorporated herein in its entirety. All publications referenced herein above are incorporated herein by reference in their entireties.

1. A configuration which is structured to be provided in an internal anatomical structure, comprising:
at least one first arrangement which includes at least one substance that, when applied to at least one section of the internal anatomical structure, has a potential to induce an inflammatory response in the at least one section; and
at least one second arrangement which is configured to receive information from the at least one section, and determine whether the inflammatory response has occurred therein.

2. The configuration according to claim 1, wherein the least one first arrangement has an array of portions into which a plurality of the at least one substance is provided.

3. The configuration according to claim 2, wherein the at least one first arrangement is structured to be placed on the surface of the at least one section, and is configured to be injected into the at least one section.

4. The configuration according to claim 1, wherein the at least one second arrangement includes at least one of an endoscopy arrangement, an systoscopy arrangement, a laporoscopy arrangement, a video-imaging probe, or an in vivo-microscopy arrangement.

5. (canceled)

6. The configuration according to claim 4, wherein the vivo-microscopy arrangement is at least one of a spectrally-encoded microscopy (SECM) arrangement, a reflectance confocal microscopy arrangement, an optical coherence tomography arrangement, a spectral-domain optical coherence tomography arrangement, an optical frequency domain imaging arrangement, a fluorescence confocal microscopy arrangement, an auto fluorescence imaging arrangement, an auto fluorescence confocal microscopy arrangement, a two-photon microscopy arrangement, a three-photon microscopy arrangement, a second harmonic microscopy arrangement, or a third harmonic microscopy arrangement.

7. The configuration according to claim 1, wherein at least one of the at least one first arrangement or the at least one second arrangement includes a balloon, a wire basket or a capsule having a shape that is configured to be swallowed.

8. The configuration according to claim 1, wherein the at least one substance includes at least one of a tumor substance, protein, cell, or molecule associated with a tumor.

9. The configuration according to claim 1, wherein the at least one second arrangement is configured to (i) determine if a cell mediated response, a humoral response, or a hypersensitivity response which occurs based on the inflammatory response, or (ii) generate at least one image of the at least one section using the information.

10. (canceled)

11. The configuration according to claim 9, wherein the at least one image includes at least one of a two-dimensional image or a three-dimensional image.

12. The configuration according to claim 1, wherein the at least one second arrangement is further configured to (i) determine whether the information is regarding at least one particular cell, or (ii) utilize a threshold of the number to determine antigen responsiveness of the at least one section.

13. The configuration according to claim 12, wherein the at least one particular cell includes at least one of an eosinophil, a lymphocyte, a plasma cell, a basophil, a mast cell, a monocyte, a macrophage, a Langerhans's cell, or a dendritic cell.

14. The configuration according to claim 12, wherein the at least one second arrangement is further configured to quantify a number of the at least one cell.

15. (canceled)

16. The configuration according to claim 1, wherein the inflammatory response is at least one of a hypersensitivity, a cell mediated immunity or a humoral immunity.

17. A configuration which obtains at least one of effects or receives information from an anatomical structure, comprising:
at least one first arrangement which includes at least one substance that, when applied to at least one section of the anatomical structure, has a potential to induce an inflammatory response in the at least one section; and
at least one microscopy second arrangement which is configured to receive information from the at least one section, and determine whether the inflammatory response has occurred therein.

18. The configuration according to claim 17, wherein the least one first arrangement has an array of portions into which a plurality of the at least one substance is provided.

19. The configuration according to claim 18, wherein the at least one first arrangement is structured to be placed on the at least one surface of the section, and is configured to be injected into the at least one section.

20. The configuration according to claim 17, wherein the at least one second arrangement includes a video-imaging probe or an in vivo arrangement.

21. (canceled)

22. The configuration according to claim 20, wherein the vivo-microscopy arrangement is at least one of a spectrally-encoded microscopy (SECM) arrangement, a reflectance confocal microscopy arrangement, an optical coherence tomography arrangement, a spectral-domain optical coherence tomography arrangement, an optical frequency domain imaging arrangement, a fluorescence confocal microscopy arrangement, an auto fluorescence imaging arrangement, an auto fluorescence confocal microscopy arrangement, a two-photon microscopy arrangement, a three-photon microscopy arrangement, a second harmonic microscopy arrangement, or a third harmonic microscopy arrangement.

23. The configuration according to claim 17, wherein at least one of the at least one first arrangement or the at least one second arrangement includes a balloon, a wire basket or a capsule having a shape that is configured to be swallowed.

24. The configuration according to claim 17, wherein the at least one substance includes at least one of a tumor substance, protein, cell, or molecule associated with a tumor.

25. The configuration according to claim 17, wherein the at least one second arrangement is configured to (i) determine if a cell mediated response, a humoral response, or a hypersensitivity response which occurs based on the inflammatory response, (ii) generate at least one image of the at least one section using the information, or (iii) determine whether the information is regarding at least one particular cell, (iv) utilize a threshold of the number to determine antigen responsiveness of the at least one section.

26. (canceled)

27. The configuration according to claim 26, wherein the at least one image includes at least one of a two-dimensional image or a three-dimensional image.

28. (canceled)

29. The configuration according to claim 23, wherein the at least one particular cell includes at least one of an eosinophil, a lymphocyte, a plasma cell, a basophil, a mast cell, a monocyte, a macrophage, a Langerhans's cell, or a dendritic cell.

30. The configuration according to claim 23, wherein the at least one second arrangement is further configured to quantify a number of the at least one cell.

31. (canceled)

32. The configuration according to claim 17, wherein the inflammatory response is at least one of a hypersensitivity, a cell mediated immunity or a humoral immunity.
